(19) United States
(12) Patent Application Publication
(10) Pub. No.: US 2007/0020228 A1
(43) Pub. Date: Jan. 25, 2007

(54) Method of using a biosealant device

(76) Inventor: Terry N. Williams, Raleigh, NC (US)

Correspondence Address: COATS & BENNETT, PLLC, P O BOX 5, RALEIGH, NC, 27602, US

(21) Appl. No.: 11/455,030

(22) Filed: Jun. 16, 2006

Related U.S. Application Data
(60) Provisional application No. 60/701,832, filed on Jul. 22, 2005.

(51) Int. Cl. A61K 31/765; A61K 31/77

(52) U.S. Cl. 424/78.38; 424/731

(57) ABSTRACT

Currently used techniques for sealing impaired tissue following medical procedures are not optimal. Manual compression, though effective, is time consuming and current sealing products use animal proteins, which can cause immunological reactions and disease. Therefore, a desirable product is one that easily and effectively seals a wound in tissue without side effects. Lipids that adhere to tissue and expand in size when applied are well suited for use as a biosealant device. Lipids, which are solid at sub-physiological temperature and exhibit a phase change at or about physiological temperature to adhere and expand within the wound are desirable for use in a biosealant device. One such class of lipid that is naturally well suited for this use are cubic phase forming monoglycerides. However, other lipids in their natural or modified states can also be used.

### RELATED APPLICATIONS

This application claims priority under 35 U.S.C. § 119(e) from the following U.S. provisional application: Application Ser. No. 60/701,832 filed on Jul. 22, 2005. That priority application is incorporated by reference herein.

### BACKGROUND

Effective closure of vascular puncture wounds and other voids in tissue following interventional or diagnostic procedures is essential. Although several commercially approved closure products are on the market today, manual compression remains the gold standard due to safety and efficacy concerns. A safe mechanism using a sealant that enhances the benefits of manual compression is extremely desirable.

Most currently used closure devices include the use of human or animal-derived proteins that promote the coagulatory cascade, including fibrinogen, thrombin and collagen. Since these proteins are isolated directly from animals, they carry the risk of causing subsequent immunological reactions and infectious disease. Production of products with animal-derived proteins necessitates their limited supply, expense and inconsistent quality. Therefore, most currently used closure devices have significant drawbacks to their use, which make them less desirable than performing manual compression.

Alternatively, a highly desirable closure product is one, which safely and effectively enhances manual compression without drawbacks like immune reactivity and infectious disease transmission that are normally associated with animal-derived protein products.

### SUMMARY

Currently used techniques for sealing impaired tissue following medical procedures are not optimal. Manual compression, though effective, is time consuming and current sealing products use animal proteins, which can cause immunological reactions and disease. Therefore, a desirable product is one that easily and effectively seals a wound in tissue without side effects.

A substance that has a solid state until physiological temperature of at or around 37° C. is reached allows the safe and efficacious delivery of the biosealant in the vascular tract down to the arteriotomy site of the vascular procedure without possible accidental intravascular introduction causing potential lower extremity blockage.

Lipids, which are solid at sub-physiological temperature and exhibit a phase change at or about physiological temperature to a liquid phase then to a cubic phase, which adheres to and expands within the wound are desirable for use as a biosealant device. One such group of lipids that is naturally well suited for this use are cubic phase forming monoglycerides. However, other lipids in their natural or modified states can also be used.

### BRIEF DESCRIPTION OF DRAWINGS

FIG. 1 is a section perspective view illustrating the site for use of the biosealant device according to one embodiment.

FIG. 2 is a section perspective view illustrating the placement of the biosealant device at the site of use according to one embodiment.

FIG. 3 is a section perspective view illustrating the biosealant device at the site of use according to one embodiment.

### DETAILED DESCRIPTION

Certain compounds with phase change properties may be used as biosealant devices to close tissue tracts, vascular puncture wounds and other voids in tissue following interventional or diagnostic procedures. These compounds are referred to herein biosealant devices. The biosealant device described herein exhibits a sold phase below normal body temperature, and changes to a liquid phase at or near body temperature. The solid phase is desirable for safety to prevent intravascular intrusion. In the liquid phase, the biosealant device absorbs body fluid and changes to the cubic phase thus expanding to seal tissue tracts. Some biosealant devices may also acquire adhesive properties in the cubic phase and adhere to surrounding tissue. The adhesive properties are not, however, attributable to polymer cross-linking. The biosealant device should not possess coagulation properties, and should be non-immunogenic.

Some amphiphilic lipids have natural properties, which make them useful as biosealants, while others can be modified and enhanced to make them more suitable for use as biosealants. Monoglycerides is one such class of lipids that are particularly well suited for use as a biosealant device because they naturally exhibit a phase change near body temperature. Lipids can safely and effectively be used as a non-immunogenic biosealant device to augment or support a hole, incision, puncture or defect in tissues and to thereby enhance manual compression. Since the lipid compound is non-thrombogenic, clot formation at the site is not enhanced and natural healing mechanisms occur as they would with manual compression alone. The lipids are then absorbed naturally into the body as the wound heals. Unlike animal derived proteins, lipids are not immunogenic and pose no risk of causing infectious disease.

The phase transition of the biosealant device promotes a sealing mechanism in three steps:
1. Rapid, localized absorption of water and small aqueous solutes promotes enhanced vasoconstriction.
2. Lipids adhere to surrounding tissue.
3. Cubic phase expansion of lipids seals the site of injury.

In one embodiment, a lipid biosealant device may comprise monoglycerides of saturated and unsaturated (cis and trans) fatty acids with the fatty acid in the one or two position on the glycerol backbone such as: glycerol monooleate, glycerol monostearate, glycerol monopalmitin, glycerol monolaurate, glycerol monocaproate, glycerol monolinoleate, glycerol monolinolenate, glycerol monomyristate and glycerol monoarachidonate. The device may also comprise diglycerides and/or triglycerides with the fatty acids in all permissible combinations on the glycerol backbone. In another embodiment, the lipid biosealant device may comprise a phospholipid such as lysophosphosphatidylcholine, lysophosphaidylethanolamine, lysophosphatidic acid, lysophosphatidylserine phosphatidylcholine, phosphatidylethanolamine, phosphatidylserine, and phosphatidic acid.

Biologically active agents can be added to the lipid biosealant device including drugs or other suitable substances that provide local or systemic biological, physiological or therapeutic effect in the body of the human or animal. Examples of useful biologically active agents include agents, or metabolic precursors thereof, that prevent infection, promote the growth, functioning and survival of cells and tissues or provide analgesic effect.

Lipids can be modified for optimal therapeutic use in a biosealant device by altering the molecular structure or by addition of agents to modify certain chemical or physical properties. For example, the molecular structure of the lipid may be changed to give the molecule an optimal phase transition temperature for use in a biosealant device at biological temperatures (34-37° C.). Alternatively, agents may be added to lipids to give the biosealant device resiliency to physical force or other desired physical characteristics for optimal use. Release rate and absorption rate modification agents may also be used to control the release of agents into the body and the absorption of the biosealant device. Additionally, increased solubility and delivery of agents may be modified by the addition of a carrier agent to the biosealant device. One embodiment may also include the addition of one or more compounds to make the biosealant device radio-opaque.

Some molecules, which may be useful in the biosealant device of the present application to alter physical properties, are polyethoxylated caster oil, polyoxyethylene alkyl ethers, polyoxyethylene ethers, polyoxyethylene fatty acid esters, polyoxyethylene stearates and sorbitan esters.

Some carrier molecules which may be useful in the biosealant device of the present application for delivery of active substances are PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG40 stearate, PEG-100 stearate, PEG-20 dilaurate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate monoglycerides, PEG-6 caprate/caprylate diglycerides, PEG-8 caprate/caprylate monoglycerides, PEG-8 caprate/caprylate diglycerides, polyglyceryl-10 laurate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, polyglyceryl-10 oleate, Tween 40, Tween 60, sucrose monostearate, sucrose monolaurate, sucrose monopalmitate, PEG 10-100 nonyl phenol series, PEG-35 castor oil, PEG-40 hydrogenated castor oil, PEG-60 corn oil, PEG-6 caprate/caprylate monoglycerides, PEG-6 caprate/caprylate diglycerides, PEG-8 caprate/caprylate monoglycerides, PEG-8 caprate/caprylate diglycerides, polysorbate 20, polysorbate 80, tocopheryl PEG-1000 succinate, a poloxamer, PEG-20 laurate, PEG-20 oleate, PEG-35 castor oil, PEG-40 palm kernel oil, PEG-40 hydrogenated castor oil, PEG-60 corn oil, polyglyceryl-10 laurate, PEG-6 caprate/caprylate monoglycerides, PEG-6 caprate/caprylate diglycerides, PEG-8 caprate/caprylate monoglycerides, PEG-8 caprate/caprylate diglycerides, polysorbate 20, polysorbate 80, POE-9 lauryl ether, POE-23 lauryl ether, POE-10 oleyl ether, sucrose monostearate, sucrose monolaurate, and the poloxamer series PEG 15-100 octyl phenol.

### EXAMPLES

### Example 1

### Sealing Arteriotomies And Venotomies

Interventional and diagnostic procedures requiring vascular incisions and punctures such as arteriotomies and venotomies during angioplasty and stent procedures can be sealed using a lipid biosealant device. The biosealant device in solid phase can be applied to a vascular puncture site alone or in combination with other devices, procedures or device to close the opening. For example, the biosealant device could be used along with a vascular clamp or other physical closure device to seal the site of tissue injury. As the biosealant device exhibits a phase change, it expands and adheres to the surrounding tissue to seal the void in tissue.

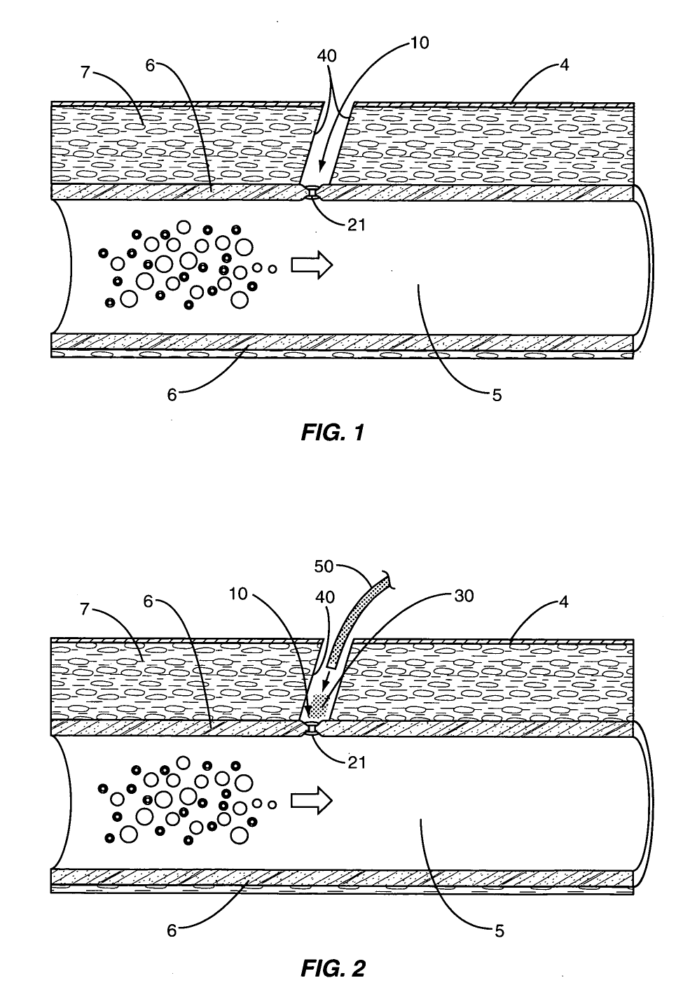

FIG. 1 illustrates the site of use for the lipid biosealant device to seal an arteriotomy site according to one embodiment. A tissue tract 40 is formed through the skin 4 and surrounding adipose and support tissue 7 to the arterial wall 6. The arterial wall 6 is cut for access to the intraluminal space 5. A vascular clamp 21 is used in this embodiment to close the site of tissue injury 10 following the procedure to seal the wound in the arterial wall 6.

FIG. 2 illustrates the application of the biosealant device 30 by way of an applicator 50 to the site of tissue injury 10 according to one embodiment. The biosealant device 30 is introduced to the site of tissue injury 10 adjacent to the vascular clamp 21 though the tissue tract 40 formed through the skin 4 and adipose and support tissue 7 to the arterial wall 6.

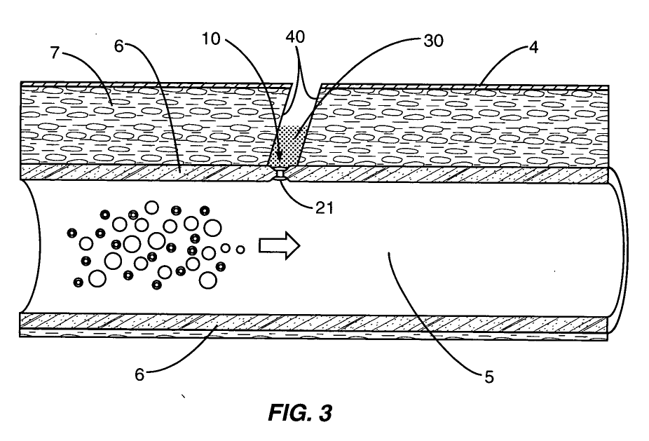

FIG. 3 illustrates the biosealant device 30 at the site of tissue injury 10 according to one embodiment. The lipids in the biosealant device 30 at their phase transition temperature enter cubic phase whereby water and other solutes are absorbed from the surrounding tissue 7 promoting dehydration and localized vasoconstriction. The biosealant device 30 adheres to the surrounding adipose and support tissue 7 and expands within the tissue tract 40 forming a tighter seal against the surrounding tissue 7, vascular clamp 21 and site of tissue injury 10.

### Example 2

### Sealing Surgical And Biopsy Tracts

Interventional and diagnostic procedures requiring punch, needle or incision biopsies of tissues or organs can be sealed using a lipid biosealant device. The biosealant device in solid phase can be applied to a biopsy site alone or in combination with other devices, procedures or compounds to close the opening. For example, the biosealant device could be used along with a clamp or other physical closure device to seal the site of tissue injury. As the biosealant device changes from solid to liquid to cubic phase, it expands and adheres to the surrounding tissue to seal the void in tissue.

### Example 3

### Biosealant Device

Biosealant devices of the present application can be made with many lipids and agents for modification of physical characteristics and biological activity. One such effective combination comprises a soy phosphatidylcholine and diacylglycerol with low levels of a polar organic co-solvent as follows:
90% phosphatidylcholine
8% diacylglycerol
2% polar organic co-solvent

The present invention may, of course, be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A method of sealing a tissue tract, said method comprising:
introducing a solid phase biosealant device into said tissue tract at a temperature lower than normal body temperature; and
allowing the biosealant device to increase to a temperature near body temperature;
said biosealant device changing from said solid phase to a liquid phase near said body temperature and from said liquid phase to a cubic phase when in contact with body fluids to close said tissue tract.

2. The method of claim 1 wherein said biosealant device expands in said cubic phase to occlude said tissue tract.

3. The method of claim 1 wherein the biosealant device compound does not possess coagulation properties.

4. The method of claim 1 wherein said biosealant device has adhesive properties in said cubic phase and adheres to surrounding tissue.

5. The method of claim 1 wherein the biosealant device induces tissue dehydration and vasoconstriction in the surrounding tissue in the lamellar/cubic phase transition.

6. The method of claim 1 wherein the biosealant device is non-immunogenic.

7. The method of claim 1 wherein the biosealant device comprises a lipid.

8. The method of claim 7 wherein the biosealant device comprises a monoglyceride of saturated or unsaturated (cis and/or trans) fatty acids.

9. The method of claim 8 wherein the biosealant device comprises one of glycerol monooleate, glycerol monostearate, glycerol monopalmitin, glycerol monolaurate, glycerol monocaproate, glycerol monolinoleate, glycerol monolinolenate, glycerol monomyristate, or glycerol monoarachidonate with the fatty acid in the one or two position on the glycerol backbone.

10. The method of claim 7 wherein the biosealant device comprises a di and tri-glyceride with the fatty acids in all permissible combinations on the glycerol backbone.

11. The method of claim 1 wherein the biosealant device further comprises a biologically-active agent to promote the growth, functioning and survival of cells and tissues, to prevent infection or to provide analgesic effect.

12. The method of claim 1 wherein the biosealant device further comprises an agent to alter physical properties of the biosealant device for increased solubility, to alter the phase transition temperature, or to increase tensile strength against vascular and physical pressures by formation of adherent matrix.

13. The method of claim 7 wherein the biosealant device comprises at least one of polyethoxylated caster oil, polyoxyethylene alkyl ethers, polyoxyethylene ethers, polyoxyetyhlene fatty acid esters, polyoxyethylene stearates and sorbitan esters.

14. The method of claim 7 wherein the biosealant device comprises a phospholipid.

15. The method of claim 14 wherein the biosealant comprises at least one of lysophosphatidylcholine, lysophosphatidylethanolamine, lysophosphatidic acid, lysophosphatidylserine phosphatidylcholine, phosphatidylethanolamine, phosphatidylserine, phosphatidic acid.

16. The method of claim 7 wherein the biosealant device comprises a principle carrier from the group consisting of PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG40 stearate, PEG-100 stearate, PEG-20 dilaurate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate monoglycerides, PEG-6 caprate/caprylate diglycerides, PEG-8 caprate/caprylate monoglycerides, PEG-8 caprate/caprylate diglycerides, polyglyceryl-10 laurate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, polyglyceryl-10 oleate, Tween 40, Tween 60, sucrose monostearate, sucrose monolaurate, sucrose monopalmitate, PEG 10-100 nonyl phenol series, PEG-35 castor oil, PEG-40 hydrogenated castor oil, PEG-60 corn oil, PEG-6 caprate/caprylate monoglycerides, PEG-6 caprate/caprylate diglycerides, PEG-8 caprate/caprylate monoglycerides, PEG-8 caprate/caprylate diglycerides, polysorbate 20, polysorbate 80, tocopheryl PEG-1000 succinate, a poloxamer, PEG-20 laurate, PEG-20 oleate, PEG-35 castor oil, PEG-40 palm kernel oil, PEG-40 hydrogenated castor oil, PEG-60 corn oil, polyglyceryl-10 laurate, PEG-6 caprate/caprylate monoglycerides, PEG-6 caprate/caprylate diglycerides, PEG-8 caprate/caprylate monoglycerides, PEG-8 caprate/caprylate diglycerides, polysorbate 20, polysorbate 80, POE-9 lauryl ether, POE-23 lauryl ether, POE-10 oleyl ether, sucrose monostearate, sucrose monolaurate, and/or the poloxamer series PEG 15-100 octyl phenol.

17. The method of claim 1 wherein the biosealant device compound is radio-opaque.
